(19) United States
(12) Patent Application Publication
(10) Pub. No.: US 2014/0338669 A1
(43) Pub. Date: Nov. 20, 2014

(54) AIR CIRCUIT ADAPTER

(71) Applicant: Shenzhen Mindray Bio-Medical Electronics Co., Ltd., Shenzhen (CN)

(72) Inventors: Hualin Zhao, Shenzhen (CN); Ji Chen, Shenzhen (CN); Weidong Zhou, Shenzhen (CN); Yunfeng Liu, Shenzhen (CN); Jian Cen, Shenzhen (CN)

(73) Assignee: Shenzhen Mindray Bio-Medical Electronics Co., Ltd.

(21) Appl. No.: 14/304,738

(22) Filed: Jun. 13, 2014

Related U.S. Application Data
(63) Continuation-in-part of application No. PCT/CN2012/086271, filed on Dec. 10, 2012.

(30) Foreign Application Priority Data
Dec. 15, 2011 (CN) 201110420112.9

(51) Int. Cl. A61M 16/08

(52) U.S. Cl. CPC A61M 16/0816; A61M 16/085
USPC 128/204.18

(57) ABSTRACT

The invention discloses an air circuit adapter that includes an external conduit, an air collector and an internal conduit. The external conduit includes a first interface and a second interface. An exterior surface of the external conduit includes a sampling interface. The interior conduit can affix inside the external conduit. The air collector can be connected with the sampling interface by a sampling air routing. The sampling air path can radially pass through the internal conduit and the external conduit.

### CROSS-REFERENCE

This application is a continuation-in-part of Patent Cooperation Treaty Application No. PCT/CN2012/086271, filed Dec. 12, 2012, which is hereby incorporated by reference.

### TECHNICAL FIELD

This disclosure relates to an air circuit adapter that can be used for connecting air.

### BACKGROUND

A low-dead-cavity type air circuit adapter is an interface conversion device that can mainly be used for an air routing connection between low tide flow pipe patients (e.g., newborns or children) and assisted breathing equipment, that is, air routing interfaces with different apertures can be connected together. Enclosed breathing air routing can be formed between patients' intubation and an assisted breathing apparatus in which patients can exchange air with breathing and anesthesia machines. Patients can ventilate through different assisted breathing equipment without changing cannulas so as to avoid patients suffering from pain. In addition, the low-dead-cavity type air circuit adapter includes an air sampling interface. A small amount of air from patients can be directed to monitoring equipment. Doctors can update patients' conditions by monitoring the patients' respiratory air composition so as to make clinical treatment more timely and effective.

A dead cavity refers to increased volume in air routing due to the insertion of the air circuit adapter. The dead cavity should be decreased to improve the effect of air sampling. In addition, a sampling interface of the air circuit adapter should be prevented from forming condensate droplets so as to avoid a sampling tube being blocked by liquid.

### SUMMARY

This disclosure provides a circuit adapter that may improve air sampling.

In one aspect, an air circuit adapter can include an external conduit, an air collector and an internal conduit. The external conduit may include a first interface and a second interface that can connect to an air path. An exterior surface of the external conduit can be equipped with a sampling interface. The internal conduit may be affixed inside the external conduit. At least a portion of the internal conduit can be inside the first interface. The air collector may be affixed inside the internal conduit and connected to the sampling interface through a sampling air path. The sampling air path can radially pass through the internal conduit and the external conduit.

In some embodiments, an interior surface of the external conduit can be equipped with a convex circular linking rib. The linking rib may enclose the internal conduit with which the linking rib can be affixed. The sampling air path may radially pass through the internal conduit, the linking rib and the external conduit in sequence.

In some embodiments, a first circular cavity surrounding the internal conduit can be formed between the internal conduit and the first interface. The first circular cavity and the second interface may be separated by the linking rib. By providing the linking rib, a small portion of the air entering into the air circuit adapter can pass through the internal conduit and the air sampling path, while the rest of the air passes through the internal conduit.

In some embodiments, the internal conduit can extend into an interior of the second interface. A second circular cavity surrounding the internal conduit may be formed between the internal conduit and the second interface. The first circular cavity and the second circular cavity may be separated by the linking rib.

In some embodiments, a front face and a rear face of the external conduit can be respectively parallel with a front face and a rear face of the internal conduit.

In some embodiments, an end of an external surface of the air collector can be equipped with circular protrusions having multiple sharp points that may be evenly distributed.

In some embodiments, the external conduit, the internal conduit, the air collector, the sampling interface and the linking rib can be integrally formed by injection molding, that is, the air circuit adapter may be formed at once using injection molding.

In some embodiments, the first interface may match with a patient's cannula, while the second interface may match with assisted breathing equipment. The first interface and the second interface can also be used to connect to other air paths that may need decreased dead volumes.

In some embodiments, an interior surface of the first interface and an exterior surface of the second interface can both be matching surfaces for interface connection and respectively provided with a taper. In other words, the interior surface of the first interface may be connected with the patient's cannula, while the exterior surface of the second interface may be connected with the assisted breathing equipment.

In some embodiments, the air circuit adapter can also include an end cap that may be detachably connected with the sampling interface. The end cap can have an open state and a closed state. The end cap may separate from the sampling interface in the open state, while sealing the sampling interface in the closed state.

In some embodiments, the end cap can include a sealing plug. The sealing plug may separate from the sampling interface when the end cap is in the open state, while inserting into and sealing the sampling interface when the end cap is in the closed state.

In some embodiments, the air circuit adapter can also include an elastically deformable connection handle that may connect to the exterior surface of the external conduit and the end cap. The connection handle can torsionally deform when the end cap is in the closed state, while the connection handle can recover its shape when the end cap is in the open state. The end cap may be independently installed, namely, the end cap can be sealingly disposed on the sampling interface when air sampling is not needed, and removed if air sampling is needed.

In another aspect, an air collector can include an interior surface and an exterior surface. The interior surface may form an air collection path for air passage, while a bottom end of the exterior surface may be equipped with circular protrusions. The air collection path can be connected with the sampling air path.

In some embodiments, the circular protrusions can include multiple sharp points.

### BRIEF DESCRIPTION OF THE DRAWINGS

FIG. 1 is a three-dimensional schematic of an air circuit adapter according to an embodiment of this disclosure.

FIG. 2 is a front view of the air circuit adapter shown in FIG. 1.

FIG. 3 is a cross-sectional view along direction A-A in FIG. 2.

FIG. 4 is a cross-sectional view along direction B-B in FIG. 2.

FIG. 5 is a three-dimensional schematic of an air circuit adapter according to another embodiment of this disclosure.

FIG. 6 is a front view of the air circuit adapter shown in FIG. 5.

FIG. 7 is a cross-sectional view along direction A-A in FIG. 6.

FIG. 8 is a cross-sectional view along direction B-B in FIG. 6.

### DETAILED DESCRIPTION

A detailed description of air routing adapters in accordance with various embodiments of the present disclosure is provided below. While several embodiments are described, it should be understood that this disclosure is not limited to any one embodiment, but instead encompasses numerous alternatives, modifications and equivalents. In addition, while numerous specific details are set forth in the following description in order to provide a thorough understanding of the embodiments disclosed herein, some embodiments can be practiced without some or all of these details. Moreover, for the purpose of clarity, certain technical material that is known in the related art has not been described in detail in order to avoid unnecessarily obscuring the disclosure.

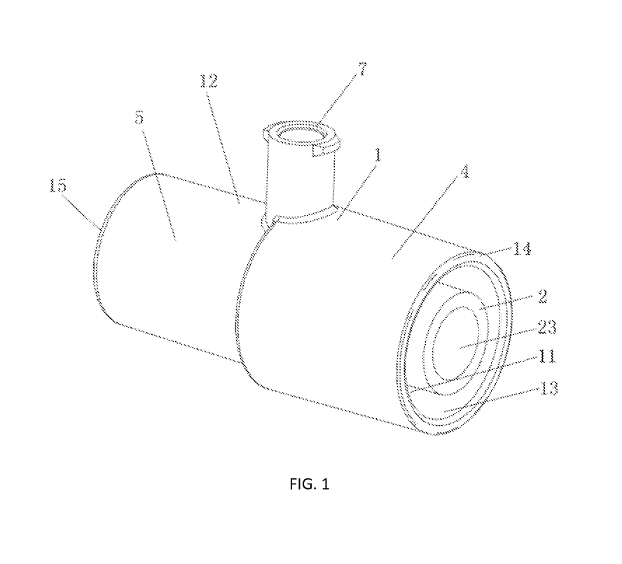

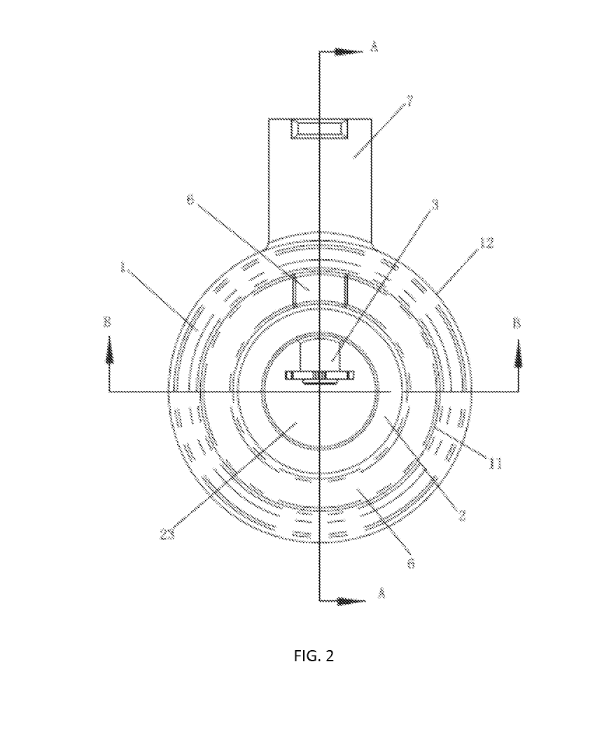

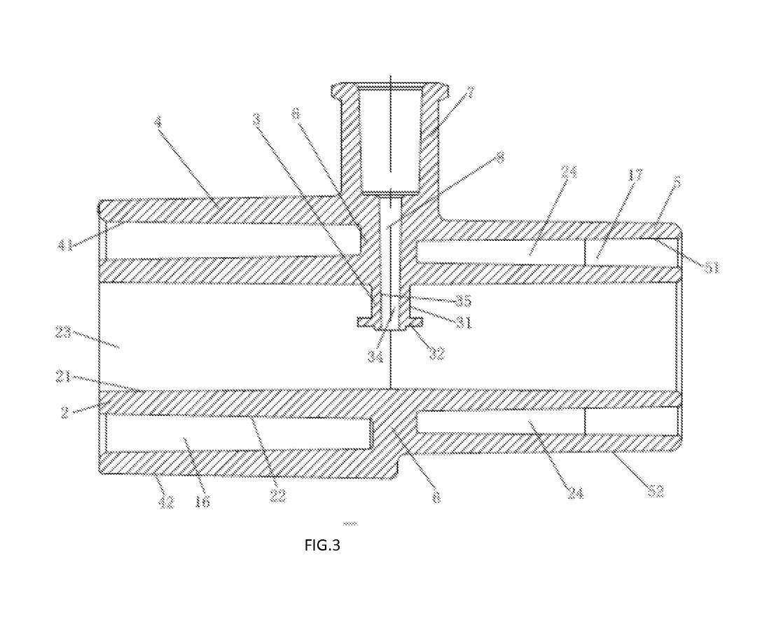

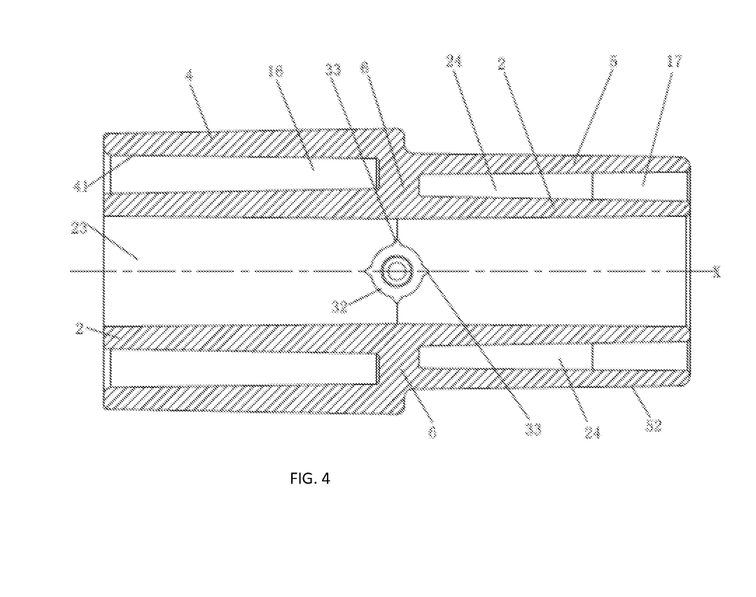

FIGS. 1 to 4 illustrate an exemplary air circuit adapter. The air circuit adapter can include an external conduit 1, an internal conduit 2 and an air collector 3. The external conduit 1 may be a structure extending through an axial direction (e.g., X-axis direction in FIG. 4), thereby forming a first internal cavity 13. A front 14 of the external conduit 1, namely one part of the external conduit 1 from the front 14 to the rear, can be a first interface 4 for matching with the cannula of patients. A rear 15 of the external conduit, namely one part of the external conduit 1 from the rear to the front 14, can be a second interface 5 matching with the assisted breathing equipment (e.g., breathing machine or anaesthesia machine). An interior surface 11 of the external conduit 1 includes a first interior surface 41 of the first interface 4 and a second interior surface 52 of the first interface 5.

As illustrated, at least some part of the internal conduit 2 can be inside the first interface 4. A first circular cavity 16 can be formed by the internal conduit 2 and the first surface 4. The internal conduit 2 could extend to the second surface 5 and form a second circular cavity 17 with the second surface 5. A linking rib 6 separates the first circular cavity 16 and the second circular cavity 17. Multiple reinforcing ribs 24 distributed inside the second circular cavity 17 can connect the interior surface 11 of the external conduit 1, an external surface 22 of the internal conduit 2 and a linking rib 6 together. In another embodiment, the internal conduit 2 could lie inside the first surface 4 instead of extending to the interior of the second interface 5. The first circular cavity 16 can separate from the second interface 5 by the linking rib 6 so as to cut off the connection of the first circular cavity 16 and the second surface 5.

As illustrated, an external interface 12 of the external conduit 1 includes a sampling interface 7 that matches the sampling conduit that connects with monitoring devices. The air collector 3 can be in a second internal cavity 23 of the internal conduit 2, and can affix in the middle of the internal surface of the internal conduit 2. The second internal cavity 23 can pass though the linking rib 6 and a sampling air routing 8 that is formed by the external conduit 1. The air collector 3 is collected with the sampling interface 7 by the sampling air routing 8. The gas collector includes an interior surface 35 and an exterior surface 31. An air routing for passing air is enclosed by an air routing 34 that can connect with the sampling air routing 8.

In some embodiments, the first interior surface 41 of the first interface 4 can be connected with patient cannulas. The second interior surface 52 of the second interface 5 can be connected with the air routing interface of assisted breathing equipment. Patients can be connected with the assisted breathing equipment by patient cannulas, the first interface 4 and the second interface 5. A small amount of air can be led to the monitoring device for analysis and patient monitoring. Air can flow from patient cannulas and be sampled after passing through the second internal cavity 23 of the internal conduit 2 and the sampling air routing 8 of the air circuit adapter. The second internal cavity 23 of the internal conduit 2 can include a dead cavity that is smaller than the first internal cavity 13 of the external conduit 1.

In some embodiments, the bottom of the exterior surface 31 of the air collector 3 can include circular protrusions 32 that are equipped with multiple sharp points (e.g., four evenly distributed sharp points 33). When air from patients passes the air circuit adapter, vapor will be liquefied into droplets on the interior surface 21 of the internal conduit 2. The droplets can flow to the sharp points 33 along the external surface 31 of the air collector and the circular protrusions 32, and then can drop down from the sharp points 33 so as to be prevented from being inhaled into the sampling air routing 8.

In some embodiments, in order to facilitate cooperation with intubation patients the interior surface 41 of the first interface 4 may include tapers. Also, the interior surface 52 of the second interface 5 may include tapers. The sampling interface 7 could be set as a standard size (e.g., a standard Luer Taper) or other surface size matching with the sampling conduit.

In some embodiments, the external conduit, the internal conduit, the sampling interface, the air collector and the linking rib can be formed at once using injection molding, that is, the air circuit adapter can be integrally formed by injection molding instead of being assembled. This can be convenient for production. The air circuit adapter could be made of medical polypropylene or other medical materials.

In some embodiments, the internal conduit can extend to the first interface and the second interface at the same time. The front and the rear of the internal conduit could parallel with the front and the rear of the external conduit.

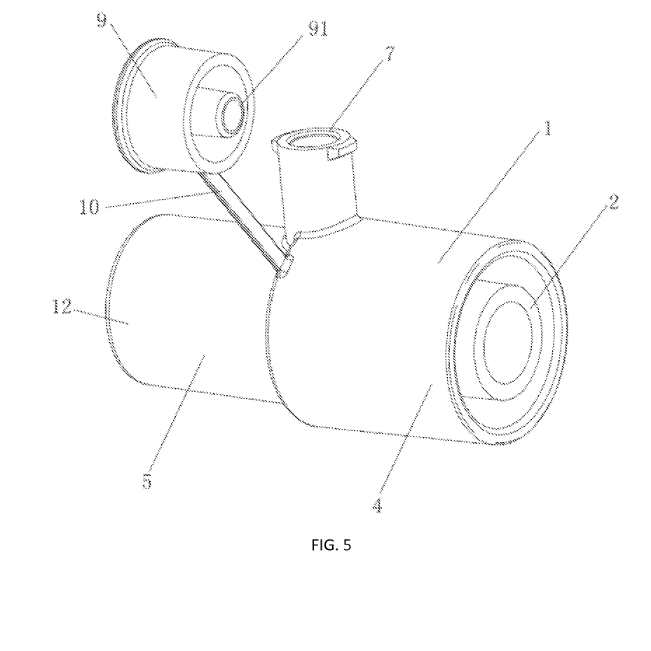

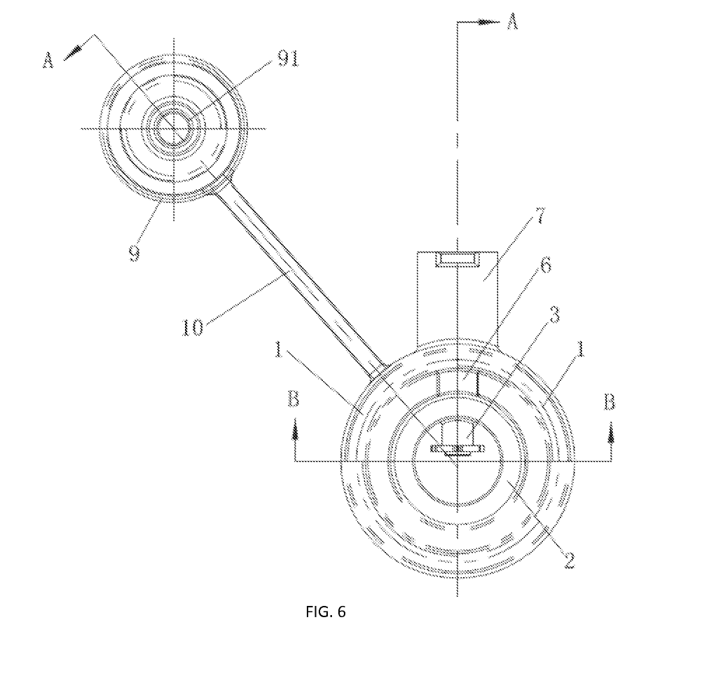

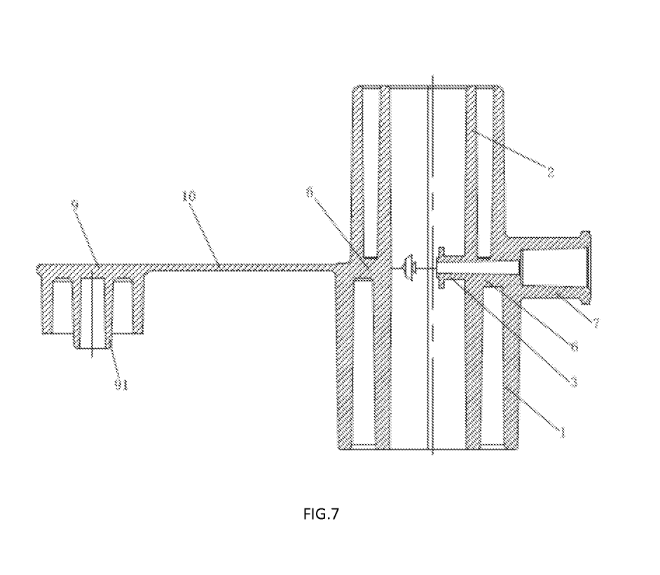

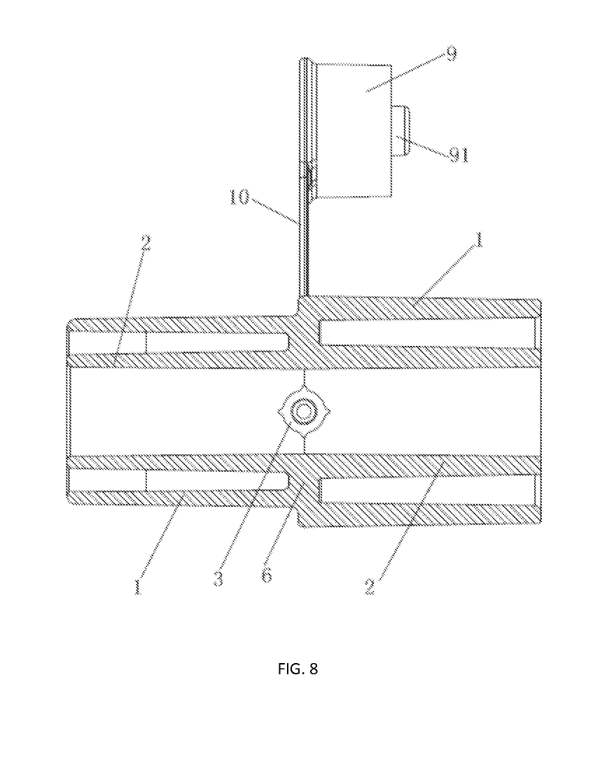

FIGS. 5 to 8 illustrate another exemplary air circuit adapter. The air adapter can include the first interface 4, the external conduit 1 of the second interface 5, the internal conduit 2, the linking rib 6, the air collector 3 and the sampling surface 7. The shape, structure and connecting relation of the external conduit 1, the internal conduit 2, the linking rib 6, the air collector 3 and the sampling surface 7 can be the same as the first exemplary air circuit adapter.

The difference compared to the first exemplary air circuit adapter is that the air circuit adapter can also include an end cap 9 that may be detachably connected with the sampling interface 7. The end cap 9 can have an open state and a closed state. The end cap 9 may separate from the sampling interface 7 in the open state, while sealing the sampling interface 7 when the end cap 9 is in the closed state. The end cap can include a sealing plug 91. The sealing plug 91 may separate from the sampling interface 7 when the end cap 9 is in the open state, while inserting into and sealing the sampling interface 7 when the end cap 9 is in the closed state.

As illustrated, the end cap 9 can be connected with the external surface 12 of the external conduit 1 by a connection handle 10. The connection handle 10 can torsionally deform when the end cap is in the closed state, while the shape of the connection handle 10 can be recovered when the end cap 9 is in the open state. In this exemplary embodiment, an angle can be formed by the connection handle 10 and the sampling surface 7 in the circle of the external conduit 1. The axis of the end cap 9 and the axis of the sampling surface 7 are vertically straight in different surfaces when in the open state; the sealing plug 91 of the end cap 9 plugs precisely into the sampling surface 7 when twisting the connection handle 10. By twisting the connection handle 10 when air sampling is not needed, the sealing plug 91 can seal the sampling surface 7. The air circuit adapter serves as the interface transition components to avoid some clinical operations (e.g., exchanging the extra interface transition components). By removing the sealing plug 91 when air sampling is needed, the connection handle 10 recovers its deformation. The air circuit adapter can be made by intensive fracture toughness medical polypropylene material and can be integrally formed by injection molding.

As illustrated, the air circuit adapter can include the external conduit. The first interface is in front of the external conduit and the external interface is in the rear of the external conduit. The external conduit can be integrally formed by injection molding. Also, the external conduit can be assembled from multiple components. The interior of the external conduit can include the internal conduit. The internal conduit not only can extend to the first interface, but can also extend to the first interface and the second interface at the same time. The front and rear of the internal conduit may be or may not be parallel with the front and rear of the external conduit.

As illustrated, the air circuit adapter can be integrally formed by injection molding or can be made from multiple parts (e.g., the air circuit adapter can be assembled from the external conduit, the sampling interface, the air collector, the linking rib and the internal conduit).

1. An air circuit adapter comprising an external conduit and an air collector, wherein the external conduit comprises a first interface and a second interface that connect to an air path, wherein the air circuit adapter further comprises an internal conduit that is affixed inside the external conduit, at least a portion of the internal conduit is inside the first interface, the air collector is affixed inside the internal conduit and connected to the interfaces through the air path, and the air path radially passes through the internal conduit and the external conduit.

2. The air circuit adapter of claim 1, wherein an interior surface of the external conduit is equipped with a convex circular linking rib that encloses the internal conduit and is affixed with the internal conduit; and the air path radially passes through the internal conduit, the linking rib and the external conduit in sequence.

3. The air circuit adapter of claim 2, wherein a first circular cavity surrounding the internal conduit is formed between the internal conduit and the first interface, and the first circular cavity and the second interface are separated by the linking rib.

4. The air circuit adapter of claim 3, wherein the internal conduit extends to an interior of the second interface, a second circular cavity surrounding the internal conduit is formed between the internal conduit and the second interface, and the first circular cavity and the second circular cavity are separated by the linking rib.

5. The air circuit adapter of claim 4, wherein a front surface and a rear surface of the external conduit respectively parallel with a front surface and a rear surface of the internal conduit.

6. The air circuit adapter of claim 4, wherein the second circular cavity is equipped with at least one reinforcing rib, and the interior surface of the external conduit, an exterior surface of the internal conduit and the linking rib are linked together by the reinforcing rib.

7. The air circuit adapter of claim 2, wherein an end of an exterior surface of the air collector is equipped with circular protrusions.

8. The air circuit adapter of claim 7, wherein the circular protrusions are equipped with multiple sharp points.

9. The air circuit adapter of claim 2, wherein the external conduit, the internal conduit, the air collector, the interfaces and the linking rib are integrally formed by injection molding.

10. The air circuit adapter of claim 1, wherein the first interface matches with a patient's cannula, and the second interface matches with assisted breathing equipment.

11. The air circuit adapter of claim 10, wherein an interior surface of the first interface and an exterior surface of the second interface are both matching surfaces for interface connection and respectively provided with a taper.

12. The air circuit adapter of claim 1, further comprising an air circuit adapter with an end cap, wherein the end cap is detachably connected with the interfaces, the end cap has an open state and a closed state, the end cap separates from the interfaces in the open state, and the end cap seals the interfaces in the closed state.

13. The air circuit adapter of claim 12, wherein the end cap comprises a sealing plug, the sealing plug separates from the interfaces in the open state, and the sealing plug inserts into and seals the interfaces in the closed state.

14. The air circuit adapter of claim 13, further comprising an air circuit adapter with an elastically deformable connection handle, wherein the connection handle connects an exterior surface of the external conduit and the end cap, and the connection handle torsionally deforms in the closed state and recovers in the open state.

15. An air collector comprising an interior surface and an exterior surface, wherein the interior surface provides an air collection circuit for air passage, and a bottom end of the exterior surface is equipped with circular protrusions.

16. The air collector of claim 15, wherein the circular protrusions are equipped with multiple sharp points.
